(19) United States
(12) Patent Application Publication
(10) Pub. No.: US 2009/0258951 A1
(43) Pub. Date: Oct. 15, 2009

(54) Use of vanillyl alcohol for the treatment of Parkinson's disease

(75) Inventors: Lun-Chung Hsu, Taipei City (TW); Zhi-Hong Wen, Kaohsiung County (TW); Kuan-Yu Lee, Taipei County (TW)

Correspondence Address: BACON & THOMAS, PLLC, 625 SLATERS LANE, FOURTH FLOOR, ALEXANDRIA, VA, 22314-1176, US

(73) Assignee: Red Pharma & Biotech Research Corporation, Pingtung County (TW)

(21) Appl. No.: 12/216,396

(22) Filed: Jul. 3, 2008

(30) Foreign Application Priority Data
Apr. 11, 2008 (TW) 097113396

(51) Int. Cl. A61K 31/085; A61P 25/00

(52) U.S. Cl. 514/717

(57) ABSTRACT

A use of Vanillyl alcohol for treatment of Parkinson's disease is disclosed in accordance with the present invention. The Vanillyl alcohol's formula is 4-hydroxy-3-methoxy-benzyl alcohol and similitude with the human neurotransmitter dopamine. The Vanillyl alcohol has dopamine receptor stimulant and which is applied for pharmaceutical preparation of treating Parkinson's disease.

### FIELD OF THE INVENTION

The present invention is generally relating to a use of Vanillyl alcohol for the treatment of Parkinson's disease.

### BACKGROUND OF THE INVENTION

Current treatment of Parkinson's disease is to modify neurotransmitter unbalance of central nervous system by means of inhibiting acetylcholine or increasing dopamine capable of slowing down symptom and maintaining patient's autonomy and activity.

However, the current medicine for the treatment of Parkinson's disease may cause cycloplegia, xerostomia, urinary retention, constipation, etc in high dosage and besides, it may cause confusion, delirium, hallucinations, etc in low dosage. Therefore, Parkinson's disease patients hesitate to take medicine because above side effects and unable to abate the symptom and obtain benefit from medical treatment.

### SUMMARY OF THE INVENTION

The primary object of the present invention is to provide a use of Vanillyl alcohol for the treatment of Parkinson's disease. Formula of the Vanillyl alcohol is 4-hydroxy-3-methoxy-benzyl alcohol and the Vanillyl alcohol having dopamine receptor stimulant is applied for pharmaceutical preparation of treating Parkinson's disease, which may prevent side effect caused by known medicine.

### DESCRIPTION OF THE DRAWINGS

FIG. 1 is a chemical structure diagram of a Vanillyl alcohol in accordance with an embodiment of the present invention.

FIG. 2 is a respective structure diagram of the Vanillyl alcohol and dopamine in accordance with an embodiment of the present invention.

FIG. 3 is a respective structure overlapped diagram of the Vanillyl alcohol and dopamine applied for observing similarity and dissimilarity of compound that cannot be exhibited under plane structure in accordance with an embodiment of the present invention.

FIG. 4 is a bar graph showing how many rotations per an hour rat rotates on 28th, 36th, 43rd and 52nd day respectively after given with amphetamine about 1 mg per 1 kg of body weight thereof via belly injection in accordance with an embodiment of the present invention.

FIG. 5 is a bar graph showing how many rotations per an hour rat rotates on 28th, 36th, 43rd and 52nd day respectively after given with amphetamine (1 mg per 1 kg of body weight thereof) via belly injection and Vanillyl alcohol (30 mg) simultaneously in accordance with an embodiment of the present invention.

FIG. 6 is a bar graph showing how many rotations per an hour rat rotates on 28th, 36th and 52nd day respectively after given with benserazide (30 mg per 1 kg of body weight thereof) via belly injection and L-DOPA (50 mg per 1 kg of body weight thereof) about 30 minutes after benserazide in accordance with an embodiment of the present invention.

FIG. 7 is a bar graph showing how many rotations per an hour rat rotates on 28th, 36th and 52nd day respectively after given with benserazide (30 mg per 1 kg of body weight thereof) via belly injection, L-DOPA (50 mg per 1 kg of body weight thereof) and Vanillyl alcohol (30 mg) 30 minutes after benserazide in accordance with an embodiment of the present invention.

### DETAILED DESCRIPTION OF THE INVENTION

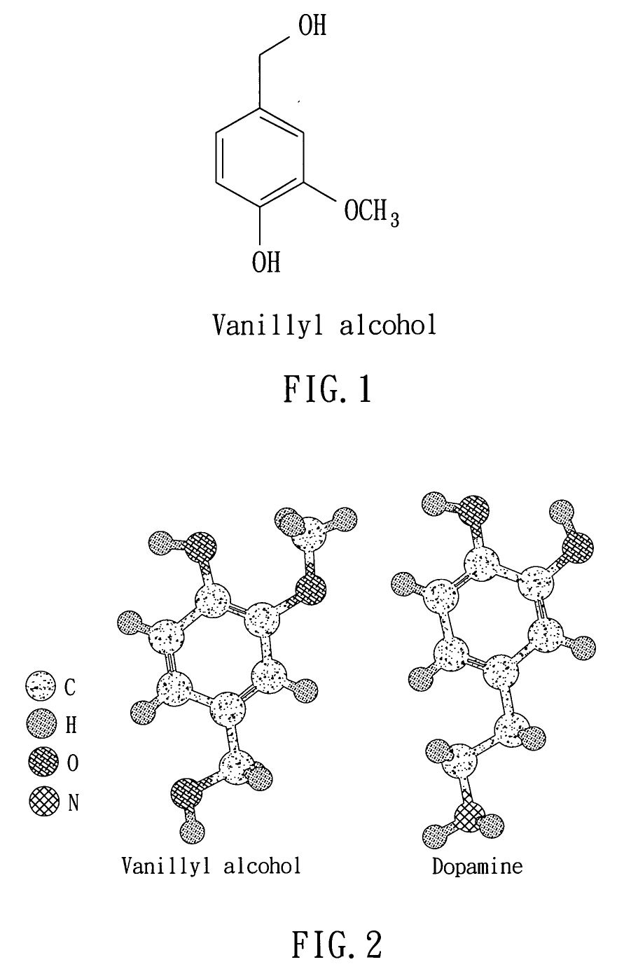

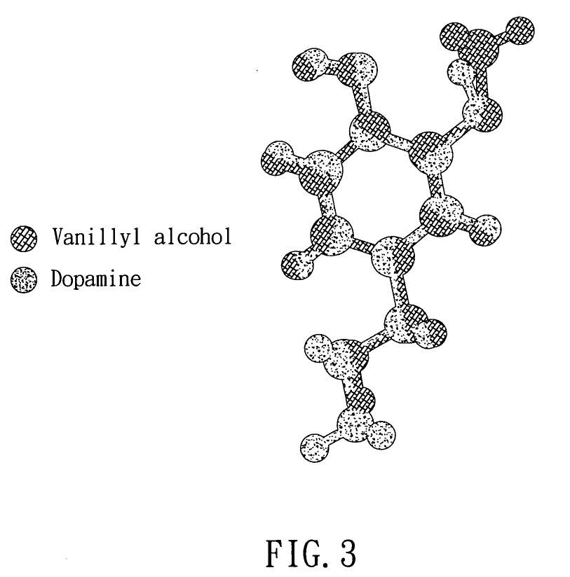

With reference to FIGS. 1, 2 and 3, a use of Vanillyl alcohol as pharmaceutical preparation for the treatment of Parkinson's disease is disclosed n accordance with an embodiment of the present invention. The Vanillyl alcohol's formula is 4-hydroxy-3-methoxy-benzyl alcohol and chemical structure thereof is shown in FIG. 1 similar to that of dopamine which is a neurotransmitter inside the human body (shown in FIGS. 2 and 3). The Vanillyl alcohol has dopamine receptor stimulant and which is applied for pharmaceutical preparation of treating Parkinson's disease. Besides, the Vanillyl alcohol is natural herbal ingredient extracted from Gastrodiae Rhizoma which is a Chinese medical material able to form a pharmaceutical composition in combination with a pharmaceutically acceptable carrier or to treat Parkinson's disease in combination with current therapeutic medicine. The Vanillyl alcohol is natural herbal ingredient capable of abating side effects which are caused after Parkinson's disease patients take medicine and suitable for long-term to take.

The following embodiment illustrates therapeutic effect of the Vanillyl alcohol against Parkinson's disease.

Make a Rat Suffer from Parkinson's Disease:

After affixing rear head of a rat with a three-dimensional tightener and anaesthetizing the rat, then inject substantia nigra located at the right side of brain of the rat with 6-OHDA (6-hydroxydopamine) to destroy substantia nigra to make the rat produce tremor, rigidity, bradykinesia etc. Next, after the rat returns to and stays in room for 2 weeks, it is given amphetamine (5 mg/1000 g) via belly injection and then starts rotating round an animal gyroscope, wherein the number of rotations will be recorded (if the number of rotations exceeds 300 rotations per an hour, it means that the amphetamine has effected on the rat).

Principle of Animal Test:

The substantia nigra of the brain of the rat is a tissue to release dopamine so that the injected amphetamine may accelerate the substantia nigra to release a large amount of dopamine. However, the substantia nigra located at the right side of brain of the rat has been destroyed by the 6-OHDA (6-hydroxydopamine) already unable to release dopamine any more, although the rat is injected with amphetamine for stimulation but the left side of body movement which is controlled by the right side of brain remains rigidity and bradykinesia. On the other hand, the substantia nigra located at the left side of brain of the rat is in normal condition without any injury so hyperkinesia occurs on the right side of body movement which is controlled by the left side of brain due to acceleration of amphetamine, that's why the rat keeps rotating for right turn (clockwise) and even uncontrollably.

Experimental Result for Testing Therapeutic Effect of Vanillyl Alcohol:

Reinforcing to Induce Symptom with Amphetamine:

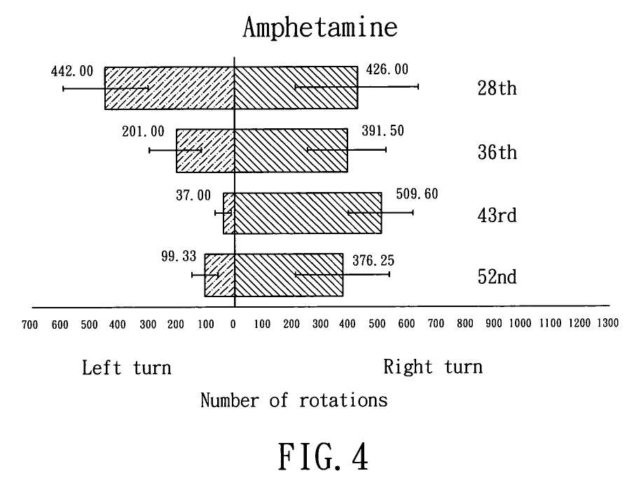

With reference to FIG. 4, the rat is given with amphetamine about 1 mg per 1 kg of body weight thereof via belly injection and rotates round an animal gyroscope on 28th, 36th, 43rd and 52nd day respectively for right turn (clockwise) and left turn (counterclockwise), in which the number of rotations will be recorded. The testing result shows obvious symptom has occurred on the rat since 36th day in accordance with record, wherein the number of rotations for left turn (counterclockwise) has decreased gradually and behavioral asymmetry between right side and left side has increased apparently.

Reinforcing to Induce Symptom with Amphetamine and Giving Vanillyl Alcohol:

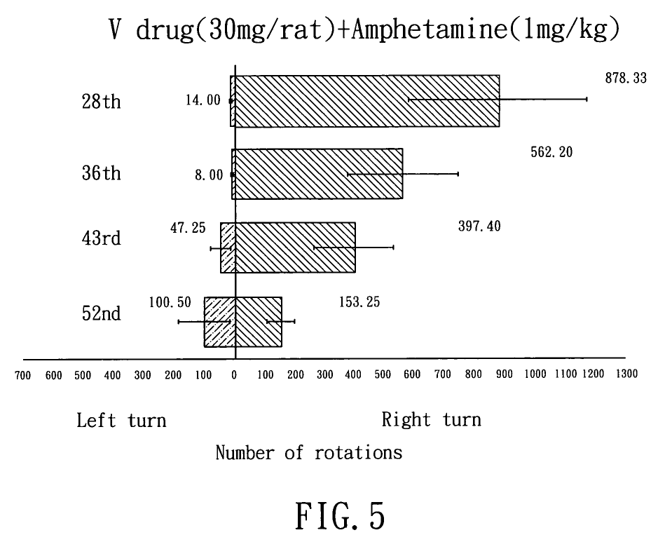

With reference to FIG. 5, the rat is given with amphetamine about 1 mg per 1 kg of body weight thereof via belly injection and further provided with Vanillyl alcohol about 30 mg. Next, the rat rotates round an animal gyroscope on 28th, 36th, 43rd and 52nd day respectively for right turn (clockwise) and left turn (counterclockwise), in which the number of rotations will be recorded. The testing result shows the number of rotations for right turn (clockwise) has decreased gradually and behavioral asymmetry between right side and left side has decreased apparently on the rat since 43rd day, especially the 52nd day is most apparently in accordance with record.

Reaction of L-DOPA:

Because dopamine cannot pass through blood brain barrier (BBB) to enter the brain through blood and L-DOPA is a typical dopamine precursor, so that L-DOPA is often applied for replacing dopamine, wherein L-DOPA may be transformed to dopamine by catalyzing dopa decarboxylase (L-aromatic amino acid decarboxylase).

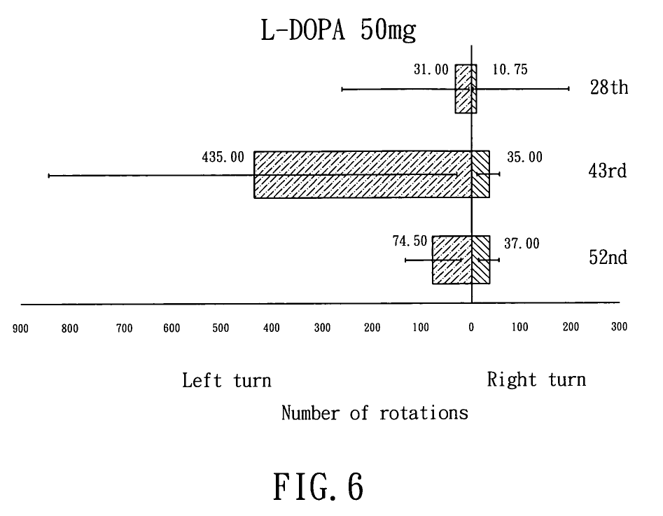

Benserazide (30 mg per 1 kg of body weight), which is applied for inhibiting circumferential tissue to take up L-DOPA as to enhance utilization rate of central nervous system, is given about 30 minutes before offering L-DOPA. With reference to FIG. 6, the rat is given with L-DOPA about 50 mg per 1 kg of body weight thereof via belly injection, in which L-DOPA is essential medicine for the treatment of Parkinson's disease. In general, there are sometimes different injury conditions for the substantia nigra between right side and left side of brain of Parkinson's disease patients, which causes the following typical symptoms, such as hand tremor at unilateral or asymmetric bilateral, bradykinesia, festination which is a gait getting progressively faster and faster and often ending in a fall, trunk flexion, or forward-flexed posture etc. However, the rat designed in this experiment suffers injury at right side merely of substantia nigra of brain thereof but left side remains normal, hence a symptom like behavioral asymmetry between right and left sides might be induced after using L-DOPA, especially the 33rd day behaves most severely.

Inducing Symptom with L-DOPA and Providing Treatment with Vanillyl Alcohol:

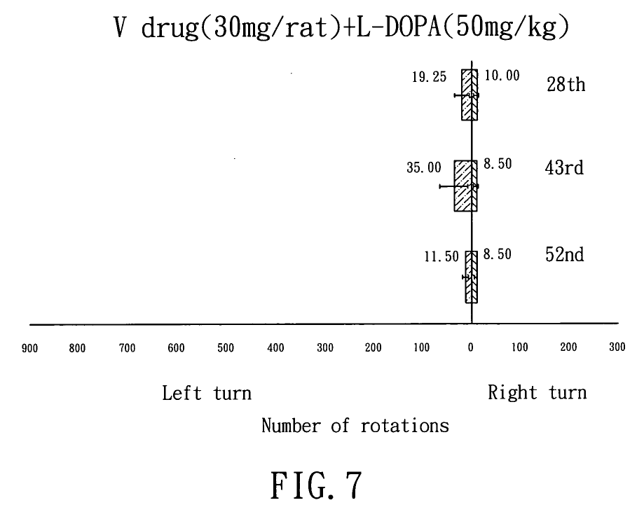

With reference to FIG. 7, the rat is given with L-DOPA about 50 mg per 1 kg of body weight thereof via belly injection and further provided with Vanillyl alcohol about 30 mg. In addition, the rat is given with benserazide (30 mg per 1 kg of body weight thereof) about 30 minutes before offering L-DOPA. Next, the rat rotates round an animal gyroscope on 28th, 36th, and 52nd day respectively for right turn (clockwise) and left turn (counterclockwise), in which the number of rotations will be recorded. All movements of the rat in the three days are almost normal in accordance with the test result, which demonstrates Vanillyl alcohol used in combination with L-DOPA designed in this experiment has therapeutic effect as well as can inhibit behavioral unbalance between right and left sides of adults induced by L-DOPA, thereby improving balance of movements of right and left sides remarkably.

While the present invention has been particularly illustrated and described in detail with respect to the preferred embodiments thereof, it will be clearly understood by those skilled in the art that various changed in form and details may be made without departing from the spirit and scope of the present invention.

What is claimed is:

1. A use of Vanillyl alcohol for treatment of Parkinson's disease, formula of the Vanillyl alcohol is 4-hydroxy-3-methoxy-benzyl alcohol and similitude with the human neurotransmitter dopamine, wherein the Vanillyl alcohol has dopamine receptor stimulant and which is applied for pharmaceutical preparation of treating Parkinson's disease.

2. The use of Vanillyl alcohol for treatment of Parkinson's disease in accordance with claim 1, wherein the Vanillyl alcohol is natural herbal ingredient.

3. The use of Vanillyl alcohol for treatment of Parkinson's disease in accordance with claim 1, wherein the Vanillyl alcohol is extracted from Gastrodiae Rhizoma which is a Chinese medical material.

4. The use of Vanillyl alcohol for treatment of Parkinson's disease in accordance with claim 1, wherein treatment dosage of the Vanillyl alcohol is more than 30 mg.

5. The use of Vanillyl alcohol for treatment of Parkinson's disease in accordance with claim 1, wherein the Vanillyl alcohol is able to form a pharmaceutical composition in combination with a pharmaceutically acceptable carrier or to treat Parkinson's disease in combination with current therapeutic medicine.
